(19) United States
(12) Patent Application Publication
(10) Pub. No.: US 2021/0098114 A1
(43) Pub. Date: Apr. 1, 2021

(54) Surgical Image Processing and Reporting System (SIPORS)

(71) Applicants: FAIZAN Mirza, Irving, TX (US); Nabeel Abdul Rahman, Raleigh, NC (US); Ayesha Badar, Raleigh, NC (US); Nabeel Balighuddin, Morrisville, NC (US); Aiman Fatima Jamadar, Navi Mumbai (IN); Nava Alam Mazumder, Cary, NC (US); Adnan Murtuza Shaikh, Raleigh, NC (US); Husaam Murtuza Shaikh, Raleigh, NC (US); Humaira Ayesha Zafiruddin, Raleigh, NC (US); Rizwan Mirza, Patna (IN)

(72) Inventors: FAIZAN Mirza, Irving, TX (US); Nabeel Abdul Rahman, Raleigh, NC (US); Ayesha Badar, Raleigh, NC (US); Nabeel Balighuddin, Morrisville, NC (US); Aiman Fatima Jamadar, Navi Mumbai (IN); Nava Alam Mazumder, Cary, NC (US); Adnan Murtuza Shaikh, Raleigh, NC (US); Husaam Murtuza Shaikh, Raleigh, NC (US); Humaira Ayesha Zafiruddin, Raleigh, NC (US); Rizwan Mirza, Patna (IN)

(73) Assignee: Innowaytors LLC, Irving, TX (US)

(21) Appl. No.: 16/583,408

(22) Filed: Sep. 26, 2019

(51) Int. Cl. G16H 30/40; G16H 30/20; G06K 9/00; H04N 5/247

(52) U.S. Cl. CPC G16H 30/40; G16H 30/20; G06K 9/00751; H04N 5/247; G06K 2209/057; G06K 9/00765; G06K 9/00335; G06K 9/00758; G06K 9/00718

(57) ABSTRACT

Surgical Image Processing and Reporting System (SIPORS) will be able to record the procedure along with transcribing the video into text through Artificial Intelligence. Artificial Intelligence is built into the system by using cameras. Through this, the system will write operative reports for doctors by going through a Writing Module in the system. Post-surgery, the doctors will be able to review the report written by the system and make edits to it. Since this is an artificial intelligence system, it will be able to learn and save the new edits the doctor makes. It will not be necessary to make this same edit again. As of now, doctors are burdened and exhausted due to the several hours spent writing the operative report. Therefore, we invented this system that reduces the time it requires to write the report.

### TECHNICAL FIELD

This invention relates to the simultaneous documentation of surgical activities of doctors, using cameras, artificial intelligence, transcription unit, and writing module. The purpose is to automate preparation of surgical processing reports and to mitigate errors.

### BACKGROUND

Much of a doctor's time goes towards writing surgical reports after the operation. Writing these operative reports is a long, time-consuming task that the doctor must complete for each individual patient. It is difficult and time consuming to write operative reports. There is no solution available to automate documentation of the surgical image or operative procedure. Using the knowledge of cameras and artificial intelligence the system will capture frames, analyze, and transcribe the surgery in an operation theatre.

### BRIEF SUMMARY

The invention works by taking in footage of the surgery via the camera. The video frames from the footage are analyzed using artificial intelligence, which will help convert the activities in the footage into readable text. The text is then used to write the surgical operative report, which is an image processing system that helps doctors write their operative reports. It records a video of the surgery, analyses the video frame-by-frame, chooses words corresponding to or matching the actions in the frames, and puts those words into sentences to finalize the surgical operative report.

### BRIEF DESCRIPTION OF THE DRAWINGS

FIG. 1 shows the direction and steps of the flow of information starting from the camera 1.

FIG. 2 shows how the report will be structured.

### DETAILED DESCRIPTION OF THE DRAWINGS

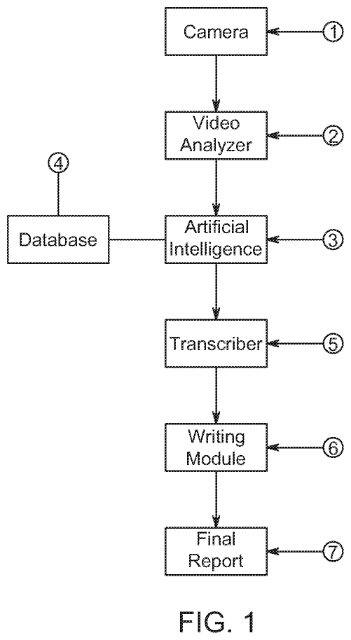

FIG. 1 shows the direction and steps of the flow of information starting from the camera 1 and to report Camera 1 is placed above the operation table to optimize the view of the subject being operated on and camera 2 will be located on the surgeon's goggles.

SIPORS will be turned on via oral command of the doctor. After being started, the system will prompt the doctor to adjust camera 1 if needed. The recording from camera 1 will be moved on to the video analyser 2, which will review the video frame by frame to figure out what the doctors are doing and what tools they are using. The database 4 will contain multiple images of tools and actions for the video analyser 2 to use and compare to. When the video analyser 2 finds a match for a frame in the database 4, it will send the code associated with that frame to the artificial intelligence 3.

The artificial intelligence 3 serves to take the incoming code from the database and translating it into words. This will then go to the transcriber 5, which will make the words from artificial intelligence 3 into humanly-readable sentences. The writing module 6 will take the sentences from the transcriber 5 and make the final report 6.

If no match is found in the database 4 for a frame when being analyzed by the video analyser 2, then there will be a note made in the document notifying the doctor that there needs to be an edit when the doctor is finalizing the report. When the edit is made, the new text inputted by the doctor will be assigned to the frame and will be added to the database.

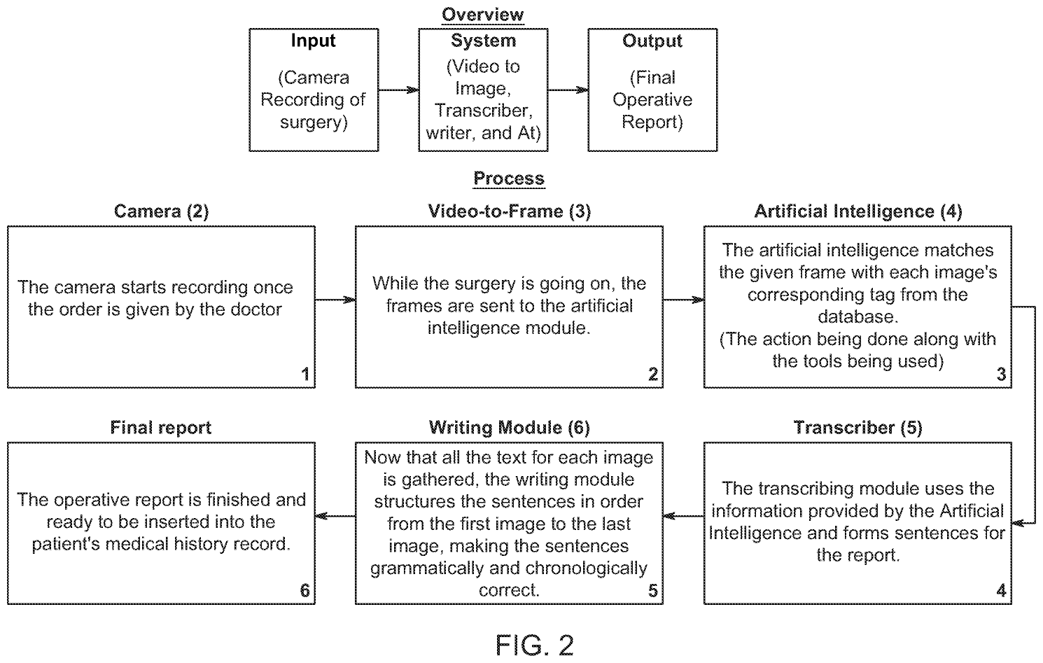

FIG. 2 shows how the report will be structured. Page 8 will be divided into two halves. The right side will contain a short clip 9 of the operation. The left side will contain the accompanying transcription that was done by the writing module 6. The blank 11 is an example of what the doctor would see when revisiting the report, signalling that needs to be edited. That edit will be remembered by database 4.

Functioning Model of the System

The Surgical Image Processing Operative Reporting System functions by taking in a video of the surgery via cameras that are placed at multiple locations. Later on, it writes the operative report, freeing up doctor's work time. The first camera will be connected to the overhead lights to record surgery from above the surgery table and the camera will be rectangular prism-shaped. The second camera will be oval-shaped and will be connected to surgeon goggles on the side where it will receive an up-close view of the surgery. The camera on the light will be 3×2×2 in. and the camera on the goggles will be 0.25×0.4×0.25 in. (Length×width×height)

The video recorded by the camera is then separated into individual frames, which are analyzed by the artificial intelligence (referred to as AI from here on out) to decode the actions of the doctors and the tools used. The AI is able to do that through a database filled with various images of different possible tools/equipment and the actions that are performed by those tools.

Each stored image in said database has a tag in the form of words in binary. The AI compares the incoming frames from the cameras to the stored images in the database. There will be two separate databases for actions and for tools/equipment. The AI will run through each database with the frame to separately identify the action being done and then the tool through which the action is done. If a match is found in the database, the AI sends the said frame and the corresponding tag to the transcription machine. The tags indicate which tool is being used or which action is being done in the given image so that the transcription module can identify it properly.

Each tag will have thousands of corresponding images from different lightings, angles, and distances. An example would be of a doctor holding a scalpel. This image would have a tag associated with it. If there is no match with any images in the database, the AI will tell the transcription machine that there is no match (this error will be elaborated later on).

The AI sends the tags with the corresponding frames to the Transcription module to be decoded from binary into an English sentence. The tag will have a unique code that the transcription machine can identify as an English sentence. Next, the sentence is sent to the writing module which formats the sentences with necessary periods and other proper punctuation as well as formats it to print in a certain manner.

The final format for the report will be in the following manner: the page will be divided into two columns, the right side of the report will have the transcription from said AI, and the left side of the report contains video clips for the matching transcription so that the Professional can watch and check if the transcription is accurate. The video will also be beneficial if/when the AI receives a frame that is not stored in its database. In such a case, transcription is skipped and only the video will be attached. This unknown part will also have a red underline with it, indicating to the professional reading it that there was something that the AI could not comprehend and attention is needed to fill in the red underline.

The professional can simply watch the associated clip on the left-hand side and fill in what the AI couldn't. This is the true reason that the use of an AI is so essential in the whole process. The AI will see what the doctor has written and learned what was going on the video. It will add it to its database as a new tool/action. this will be a never-ending cycle of the AI continuously learning new images and increasing its default database. Although this invention is specifically designed for surgery and live transcription, it can have a wide variety of uses in almost anything that requires transcription to be done for a procedure taking place. For example, a person working on a machine and needs to transcribe all actions done to the machine.

1. Surgical Image Processing and Reporting System (SIPORS) consists of (a) 2 cameras, where one rectangular prism-shaped camera on surgeon's goggles and one oval-shaped placed on surgical lights (b) AI based image processing system in order to turn recorded surgery video into images/frames (c) transcribing module where tags (many frames) are coded from binary into English sentences.

2. SIPORS's transcribing module separated from said image processing system and said artificial intelligence unit do also consists of writing module which places the words into complete sentences with proper grammar.
